(19) United States
(12) Patent Application Publication
(10) Pub. No.: US 2009/0209576 A1
(43) Pub. Date: Aug. 20, 2009

(54) PHARMACEUTICAL COMPOSITION CONTAINING CLOPIDOGREL CAMPHORSULFONATE OR POLYMORPHIC FORMS THEREOF

(75) Inventors: Won Jeoung Kim, Suwon-si (KR); Han Kyong Kim, Yongin-si (KR); Kwee Hyun Suh, Suwon-si (KR); Gwan Sun Lee, Seoul (KR)

Correspondence Address: ROTHWELL, FIGG, ERNST & MANBECK, P.C., 1425 K STREET, N.W., SUITE 800, WASHINGTON, DC, 20005, US

(73) Assignee: HANMI PHARM. CO., LTD., Hwaseong-gun, Kyungki-do (KR)

(21) Appl. No.: 12/293,357

(22) PCT Filed: Mar. 15, 2007
(86) PCT No.: PCT/KR07/01278

§ 371 Date: Sep. 17, 2008

(30) Foreign Application Priority Data
Mar. 17, 2006 (KR) 10-2006-0024610

(51) Int. Cl. A61K 31/4365; C07D 495/04; A61P 7/00

(52) U.S. Cl. 514/301; 546/114

(57) ABSTRACT

A pharmaceutical composition of the present invention which comprises clopidogrel camphorsulfonate of formula (I) or its polymorphic forms as an active ingredient is useful for treating or preventing a platelet aggregation-associated disease.

### FIELD OF THE INVENTION

The present invention relates to a pharmaceutical composition containing the salt of clopidogrel and camphorsulfonic acid or its polymorphic forms as an active ingredient.

### BACKGROUND OF THE INVENTION

Clopidogrel, methyl(+)-(S)-α-(o-chlorophenyl)-6,7-dihydrothieno[3,2-a]pyridine-5(4H)-acetate of formula (II), is a platelet-aggregation inhibitor which is effective in treating and preventing various platelet-associated vascular diseases such as stroke, cerebral arteriosclerosis, myocardial infarction, angina pectoris, arrhythmia, peripheral arteries disease and Burger's disease.

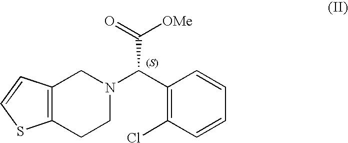

As a free base form of clopidogrel is an oil which is relatively unstable and is difficult to purify, a stable solid form of clopidogrel suitable for use in the preparation of a pharmaceutical composition has been sought, and there have been reported an acid addition salt of clopidogrel and its polymorphic forms obtained by using a pharmaceutically acceptable inorganic or organic acid.

For example, U.S. Pat. No. 4,847,265 discloses a number of acid addition salts of clopidogrel including hydrochloride, hydrobromide, hydrogen sulfate and taurocholate. In addition, U.S. Pat. No. 6,767,913, U.S. Publication Patent No. 2003/225129 and International Publication Patent No. WO 2004/081016 disclose new polymorphic forms of clopidogrel hydrogen sulfate; International Publication Patent Nos. WO 2003/006637, WO 2005/068471 and WO 2005/080890, polymorphic forms of each of clopidogrel hydrochloride and clopidogrel hydrobromide; and International Publication Patent Nos. WO 2004/072084 and WO 2004/106344, solvates and polymorphic forms of each of solid clopidogrel benzenesulfonate and toluenesulfonate.

Among the above acid addition salts of clopidogrel, the most preferred has been crystalline clopidogrel hydrogen sulfate. However, it is known that clopidogrel hydrogen sulfate employed in PLAVIX™ (Sanofi-Synthelabo Inc.), a commercial tablet composition, is not sufficiently stable. For example, it was reported that PLAVIX™ is unstable under an accelerated test condition (40° C., 75% relative humidity, for 3 months), generating significant amounts of impurities such as hydrolyzed by-products and levorotatory isomers (see [Y. Gomez et al., J. Pharm. Biomed. Anal. 34: 341-348, 2004] and [H. Agrawal et al., Talanta, 61: 581-589, 2003]).

As the stability of the active ingredient used in a pharmaceutical composition is very important in terms of shelf storage life, maintenance of its activity and suppressing undesirable side effects, there has been a need to develop an improved salt of clopidogrel.

U.S. Pat. No. 4,847,265, and International Publication Patent Nos. WO 2004/074215, WO 2004/013147 and WO 2004/106344 disclose the use of camphorsulfonic acid salt of clopidogrel, specifically, (−)-(1R)-camphor-10-sulfonic acid salt in the process of optically resolving racemic clopidogrel.

The inventors have unexpectedly found that such camphorsulfonic acid salt of clopidogrel or some polymorphic forms thereof is more stable toward moisture and heat than conventional acid addition salts.

### SUMMARY OF THE INVENTION

It is an object of the present invention to provide a pharmaceutical composition containing the salt of clopidogrel and camphorsulfonic acid or its polymorphic forms as an active ingredient.

### BRIEF DESCRIPTION OF THE DRAWINGS

The above and other objects and features of the present invention will become apparent from the following description of the invention, when taken in conjunction with the accompanying drawings which respectively show:

FIGS. 1 to 3: a powder X-ray diffraction spectrum, a differential scanning calorimeter and an FT-IR absorption spectrum of the polymorphic form A1 of clopidogrel (−)-(1R)-camphor-10-sulfonate, respectively;

FIGS. 4 to 6: a powder X-ray diffraction spectrum, a differential scanning calorimeter and an FT-IR absorption spectrum of the polymorphic form B1 of clopidogrel (+)-(1S)-camphor-10-sulfonate, respectively;

FIGS. 7 to 9: a powder X-ray diffraction spectrum, a differential scanning calorimeter and an FT-IR absorption spectrum of the polymorphic form B2 of clopidogrel (+)-(1S)-camphor-10-sulfonate, respectively;

FIGS. 10 and 11: a powder X-ray diffraction spectrum and a differential scanning calorimeter of the polymorphic form B3 of clopidogrel (+)-(1S)-camphor-10-sulfonate, respectively;

FIG. 12: time-dependent changes (%) in the assay of four acid addition salts of clopidogrel;

FIG. 13: time-dependent changes (%) in the amount of hydrolyzed impurities of four acid addition salts of clopidogrel;

FIG. 14: time-dependent changes (%) in the amount of levorotatory isomers of four acid addition salts of clopidogrel; and

FIG. 15: time-dependent changes (%) in the water content of four acid addition salts of clopidogrel.

### DETAILED DESCRIPTION OF THE INVENTION

In accordance with one aspect of the present invention, there is provided a pharmaceutical composition for treating or preventing a platelet aggregation-associated disease, which comprises clopidogrel camphorsulfonate of formula (I) or its polymorphic forms as an active ingredient:

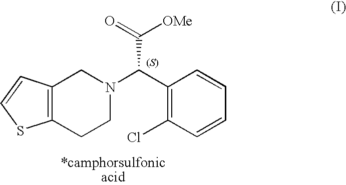

The clopidogrel camphorsulfonate of formula (I) may be (−)-(1R)-camphor-10-sulfonic acid addition salt of clopidogrel of formula (Ia) or (1S)-camphor-10-sulfonic acid addition salt of clopidogrel of formula (Ib):

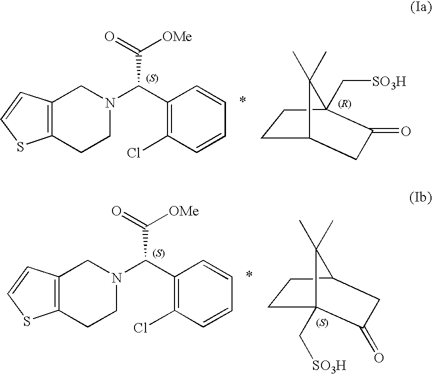

The clopidogrel (−)-(1R)-camphor-10-sulfonate of formula (Ia) and the clopidogrel (+)-(1S)-camphor-10-sulfonate of formula (Ib) may both have various forms of crystals, i.e., polymorphic forms. Specifically, the clopidogrel (−)-(1R)-camphor-10-sulfonate of formula (Ia) may have at least two polymorphic forms including polymorphic forms A1 and A2, and the clopidogrel (+)-(1S)-camphor-10-sulfonate of formula (Ib), at least three polymorphic forms including polymorphic forms B1, B2 and B3. Thus, the inventive pharmaceutical composition may comprise any of the polymorphic forms A1, A2, B1, B2 and B3, or a mixture thereof as an active ingredient.

In accordance with the present invention, there are provided polymorphic forms A1 and A2 of the clopidogrel (−)-(1R)-camphor-10-sulfonate of formula (Ia), and polymorphic forms B1, B2 and B3 of the clopidogrel (+)-(1S)-camphor-10-sulfonate of formula (Ib).

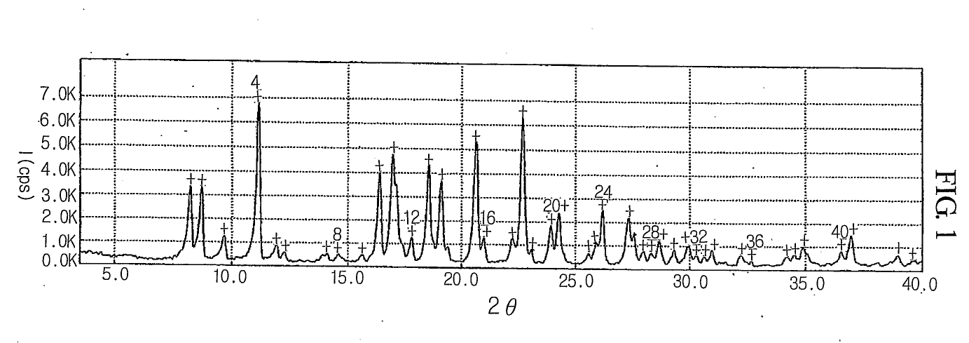

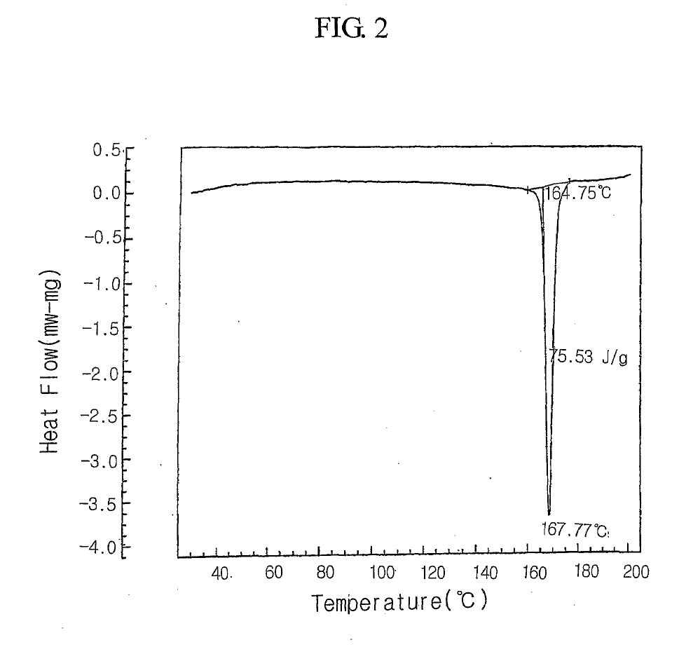

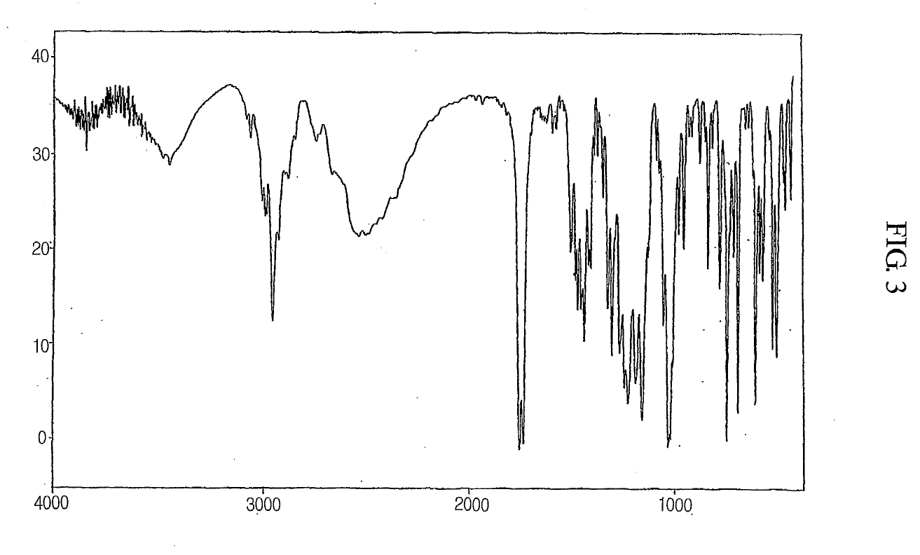

The polymorphic form A1 of the clopidogrel (−)-(1R)-camphor-10-sulfonate has the crystal form whose powder X-ray diffraction (XRD) scan shows major peaks having I/I₀ values greater than 20% (100×I/I₀>20) at 2theta (2θ) of 8.2, 8.7, 11.2, 16.4, 17.0, 18.6, 19.1, 20.6, 22.7, 23.9, 24.3, 26.2, 27.3 and 36.9 (FIG. 1). Differential scanning calorimeter (DSC) curve of the polymorphic form A1 at 10° C./min shows an absorption peak of about 75.5 J/g whose heat absorption starts at about 165° C. and reaches its maximum at about 168° C. (FIG. 2). The actually observed melting point of the polymorphic form A1 is 162 to 165° C. Further, the polymorphic form A1 is characterized by having major peaks at 2956, 1756, 1737, 1471, 1454, 1438, 1324, 1304, 1266, 1243, 1226 and 1190 cm⁻¹ in its FT-IR absorption spectrum, as shown in FIG. 3.

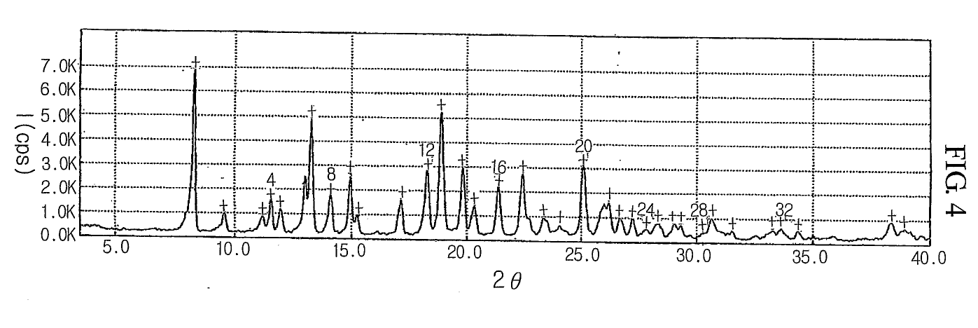

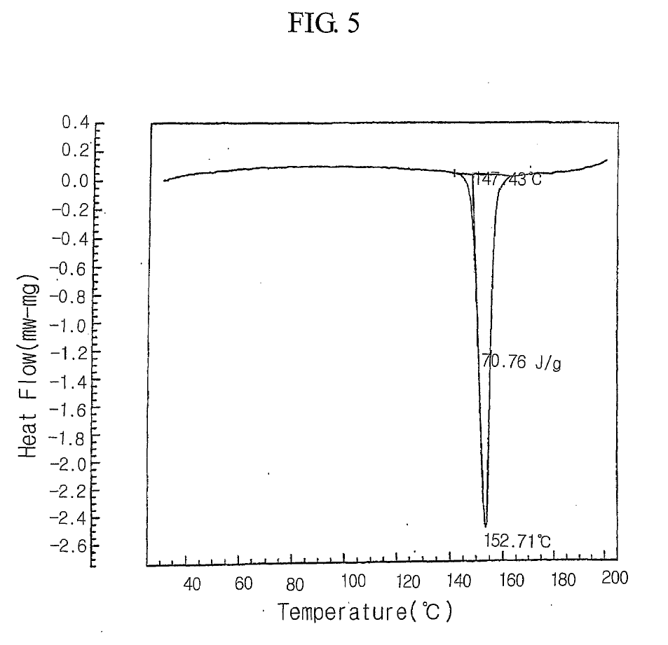

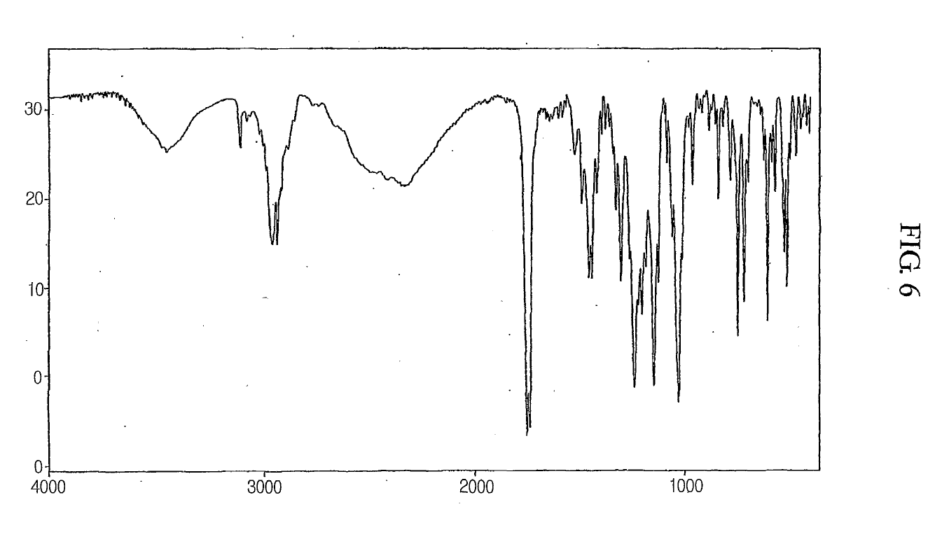

The polymorphic form B1 of the clopidogrel (+)-(1S)-camphor-10-sulfonate has the crystal form whose powder XRD scan shows major peaks having I/I₀ values greater than 20% (100×I/I₀>20) at 2θ of 8.3, 11.5, 13.0, 13.3, 14.1, 15.0, 17.1, 18.3, 18.9, 19.8, 21.4, 22.4, 25.1 and 26.0 (FIG. 4). DSC curve of the polymorphic form B1 at 10° C./min shows an absorption peak of about 70 J/g whose heat absorption starts at about 145° C. and reaches its maximum at about 151° C. (FIG. 5). The actually observed melting point of the polymorphic form B1 is 149 to 150° C. Further, the polymorphic form B1 is characterized by having major peaks at 2959, 2936, 1752, 1739, 1452, 1437, 1301, 1241, 1147, 1028, 753, 723 and 613 cm⁻¹ in its FT-IR absorption spectrum, as shown in FIG. 6.

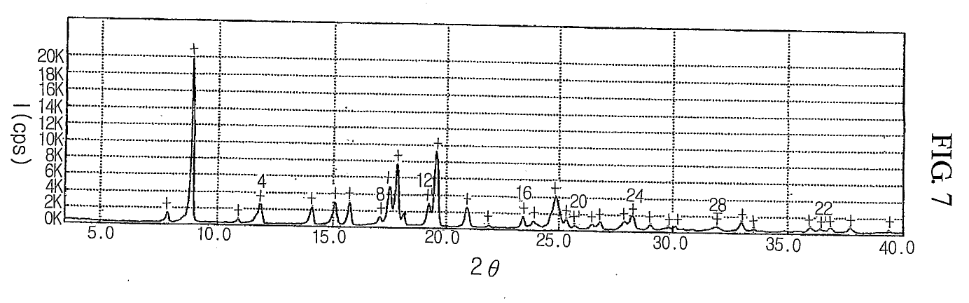

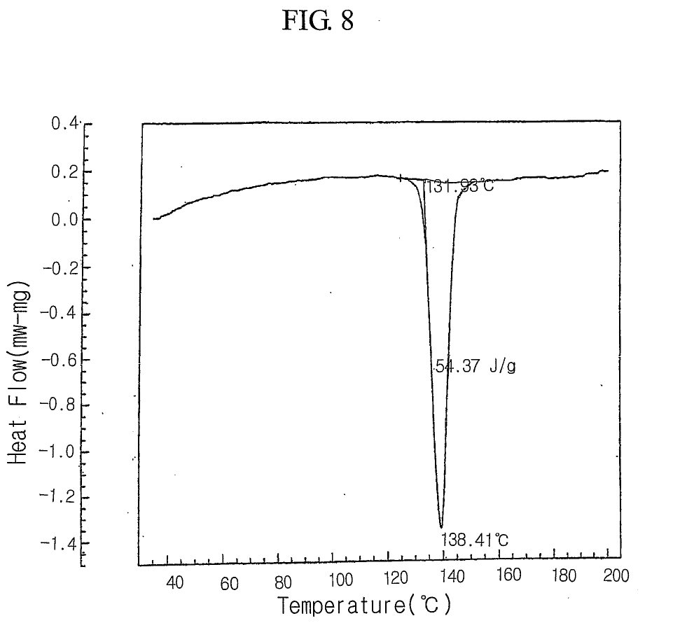

The polymorphic form B2 of the clopidogrel (+)-(1S)-camphor-10 sulfonate has the crystal form whose powder XRD scan shows major peaks having I/I₀ values greater than 10% (100×I/I₀>10) at 2θ of 7.9, 11.9, 14.1, 15.1, 15.7, 17.5, 17.8, 19.2, 19.5, 20.9 and 24.9 (FIG. 7). DSC curve of the polymorphic form B2 at 10° C./min shows an absorption peak of about 54 J/g whose heat absorption starts at about 132° C. and reaches its maximum at about 138° C. (FIG. 8). The actually observed melting point of the polymorphic form B2 is 135 to 136° C. Further, the polymorphic form B2 is characterized by having major peaks at 2959, 1748, 1479, 1446, 1322, 1301, 1233, 1201, 1145, 1024, 754, 725 and 614 cm⁻¹ in its FT-IR absorption spectrum, as shown in FIG. 9.

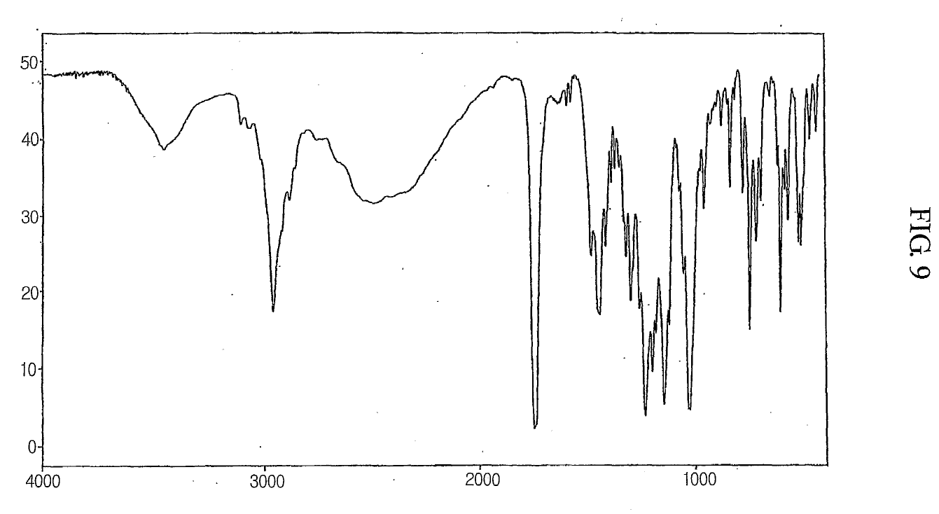

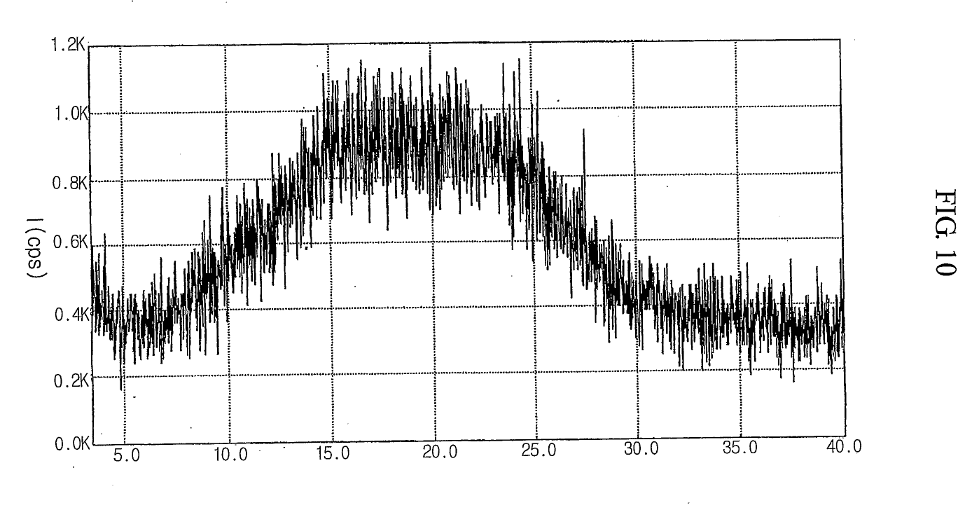

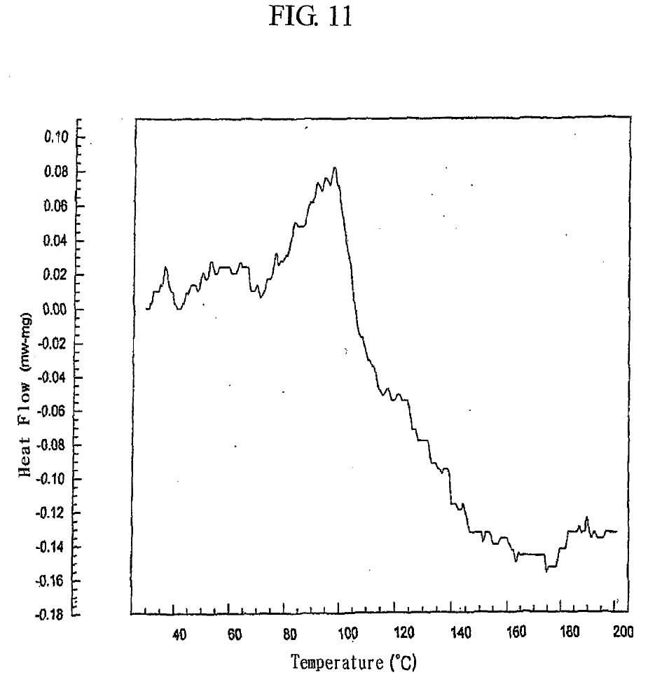

The polymorphic forms A2 and B3 are amorphous forms of clopidogrel (−)-(1R)-camphor-10-sulfonate and clopidogrel (+)-(1S)-camphor-10-sulfonate, respectively. These amorphous salts have neither prominent diffraction angles at the powder XRD spectrum nor prominent heat absorption and heat discharge peaks at the DSC, which is confirmed from the curves of FIGS. 10 and 11 which represent the powder XRD spectrum and the DSC of the polymorphic form B3, respectively.

(+)-(1S)-Camphor-10-sulfonic acid for the preparation of the clopidogrel camphorsulfonate of formula (I) is frequently employed in the formation of the acid addition salt of alkaline active ingredient, and it is referred to as “camsilate” according to INN and as “camsylate” according to USAN (see [S. M. Berge et al., J. Pharm. Sci. 66: 1, 1977], [Handbooks of Pharmaceutical Salts, ‘Properties, Selection, and Use’, P. H. Stahl] and [C. G. Wermuth Eds., 2002, Verlag Helvetica Chimica Acta, Zurich, pp 275]).

The polymorphic forms A1, B1 and B2 may be each prepared by reacting clopidogrel (dextro-rotatory clopidogrel free base) with camphorsulfonic acid, i.e., (−)-(1R)-camphor-10-sulfonic acid or (+)-(1S)-camphor-10-sulfonic acid, in an organic solvent. Representative examples of the organic solvent which may be used in the present invention include diethyl ether, diisopropyl ether, methyl t-butyl ether, methyl acetate, ethyl acetate, n-propyl acetate, isopropyl acetate, n-butyl acetate, isobutyl acetate, acetone, methyl ethyl ketone, methyl isobutyl ketone, tetrahydrofuran, 1,4-dioxane, isopropanol and a mixture thereof.

The organic solvent may be employed in an amount ranging from 1 to 50 by ml volume, preferably 3 to 20 by ml volume, based on 1 g weight of clopidogrel. It is preferred that when diethyl ether, diisopropyl ether or methyl t-butyl ether is used together with other organic solvents, its volume does not exceed 80% of the total volume of the solvent. Further, camphorsulfonic acid, i.e., (−)-(1R)-camphor-10-sulfonic acid or (+)-(1S)-camphor-10-sulfonic acid, may be employed in an amount ranging from 0.9 to 1.2 moles based on 1.0 mole of clopidogrel.

The reaction and the crystallization of the salt may be performed at a temperature ranging from −10° C. to the boiling point of the solvent for a period ranging from 0.1 to 24 hours. Preferably, after precipitation formation, the resulting reaction mixture may be allowed to be cooled to a temperature ranging from −10° C. to room temperature and to be stirred for a period ranging from 1 to 24 hours.

The precipitates thus formed may be filtered and washed with the solvent used in the reaction or the crystallization. The precipitates are dried using an inert gas such as air and nitrogen under an atmospheric pressure or under a reduced pressure at a temperature ranging from room temperature to 70° C. If necessary, the polymorphic forms A1, B1 and B2 may be each further purified by recrystallization under the organic solvent.

The polymorphic form A2 may be prepared by dissolving the polymorphic form A1 in at least one organic solvent selected from the group consisting of methanol, ethanol, acetone, acetonitrile, dichloromethane and chloroform, spray drying the solution or removing the solvent therefrom under a reduced pressure, and grinding the resulting residue. So may be prepared the polymorphic form B3 from the polymorphic form B1 or B2.

When the polymorphic forms A1 and B1 are revealed to an accelerated condition of 60° C. and 75% relative humidity for a period of over 28 days, they almost maintain their original water contents, thereby being less hygroscopic, and very stable against moisture and heat. In addition, the polymorphic forms A1 and B1 show satisfactory water-solubilities of around 4.0 mg/ml when measured in a saturated aqueous solution, which represents their effective dissolution from pharmaceutical compositions containing same. Even though clopidogrel has a relatively low optical purity of 90.0% ee (enantiomeric excess), the desired clopidogrel salt obtained therefrom may have a significantly high optical purity of at least 98.5% ee.

Thus, the clopidogrel camphorsulfonate of formula (I) and its polymorphic forms may be employed each independently or in a mixed form as an active ingredient in the preparation of a pharmaceutical composition. A pharmaceutical composition comprising the clopidogrel camphorsulfonate or its polymorphic forms can be useful for the treatment and prevention of a platelet aggregation-associated disease.

Representative examples of the platelet aggregation-associated disease include stroke, cerebral arteriosclerosis, myocardial infarction, angina pectoris, arrhythmia, peripheral arteries disease and Burger's disease.

The inventive pharmaceutical composition comprising the clopidogrel camphorsulfonate or its polymorphic forms as an active ingredient may be administered via the oral route, and, thus, the inventive pharmaceutical composition may be in the form of solutions, suspensions, tablets, pills, capsules, powders and the like.

The pharmaceutical composition according to the present invention may be formulated together with pharmaceutically acceptable carriers, if necessary.

Examples of suitable carriers are excipients such as starch, sugar and mannitol; filling agents or increasing agents such as calcium phosphate and silica derivatives; binding agents such as cellulose derivatives of carboxymethylcellulose or hydroxypropylcellulose, gelatin, arginic acid salt and polyvinylpyrrolidone; lubricating agents such as talc, magnesium or calcium stearate, hydrogenated castor oil and solid polyethylene glycol; disintegrants such as povidone, croscarmellose sodium and crospovidone; and surfactants such as polysorbate, cetyl alcohol and glycerol monostearate. Further, various pharmaceutical compositions comprising a specific amount of an active ingredient together with or without such additives may be prepared in accordance with any of the conventional procedures (see [Remington's Pharmaceutical Science, Mack Publishing Company, Easton, Pa., 19^{th} Edition, 1995]).

The inventive pharmaceutical composition may contain the clopidogrel camphorsulfonate or its polymorphic forms in an amount ranging from 0.1 to 95% by weight, preferably 1 to 70% by weight, based on the total weight of the composition.

The clopidogrel camphorsulfonate of formula (I) may be orally administered to a subject in a dose ranging from 1 to 2,000 mg/60 kg weight, preferably 25 to 600 mg/60 kg weight per day with one portion or divided portions.

The following Examples are given for the purpose of illustration only, and are not intended to limit the scope of the invention.

The analysis conditions of HPLC employed in Examples are listed below.

### Condition A: For the Measurement of the Assay of Acid Addition Salt of Clopidogrel

Column: Kromasil C18, 5 μm (250 mm×4.6 mm)

Detector: 220 nm

Flow rate: 1.5 ml/min

Mobile phase: Na₂HPO₄—NaH₂PO₄ buffer solution:TBF:CH₃CN=5:2:3 (v/v)

### Condition B: For the Measurement of the Amount of Hydrolyzed Impurities of Acid Addition Salt of Clopidogrel

Column: Capcellpak C18 MG, 5 μm (250 mm×4.6 mm)

Detector: 210 nm

Flow rate: 1.0 ml/min.

Mobile phase: KH₂PO₄ buffer solution/CH₃CN (70/30):KH₂PO₄ buffer solution/CH₃CN (30/70)=0:100→1100:0 (v/v, gradient elution)

### Condition C: For the Measurement of the Optical Purity of Acid Addition Salt of Clopidogrel

Column: Chiralpak AD, 5 μm (250 mm×4.6 mm)

Detector: 210 nm

Flow rate: 1.0 ml/min.

Mobile phase: n-hexane:isopropanol=90:10 (v/v)

In the following Examples, employed was clopidogrel free base which had been prepared by the methods disclosed in U.S. Pat. No. 4,847,265 and International Publication Patent No. WO 2005/087779.

### Comparative Example 1

### Preparation of Clopidogrel Hydrogen Sulfate

Clopidogrel hydrogen sulfate as a crystalline form II was prepared according to the method similar to that disclosed in U.S. Pat. No. 6,429,210 from clopidogrel free base having an optical purity of 98.5% ee.

m.p.: 176˜177° C. (calculated value 176° C.)

water (Karl-Fisher titrator): below 0.1%

optical purity (HPLC, condition C): 98.6% ee

### Comparative Example 2

### Preparation of Clopidogrel Benzenesulfonate

Clopidogrel benzenesulfonate as a crystalline form III was prepared according to the method similar to that disclosed in International Publication Patent No. 2005/0203122 from clopidogrel free base having an optical purity of 98.5% ee.

m.p.: 134˜136° C. (calculated value 135-138° C.)

water (Karl-Fisher titrator): below 0.1%

optical purity (HPLC): 99.3% ee

### Example 1

### Preparation of clopidogrel (−)-(1R)-camphor-10-sulfonate (Polymorphic Form A1)

20 g of clopidogrel free base having an optical purity of 98.5% ee was dissolved in 200 ml of acetone, and 14.5 g of (−)-(1R)-camphor-10-sulfonic acid was added thereto. Then, the mixture was stirred at room temperature for 4 hours. The precipitates formed were filtered, washed with cool acetone and dried at 50° C., to obtain 1.48 g of the title compound (yield: 86%) as an white crystal.

m.p.: 165-167° C.

water (Karl-Fisher titrator): below 0.1%

specific rotatory power: [α]_{D}²⁰+25.0 (c=1, methanol)

optical purity (HPLC, condition C): above 99.6% ee

IR (KBr, cm⁻¹): 2956, 1756, 1737, 1471, 1454, 1438, 1324, 1304, 1266, 1243, 1226, 1190 (FIG. 3)

DSC (10° C./min): starting point about 165° C., lowest point about 168° C. (heat absorption about 75.5 J/g, FIG. 2)

The result of powder X-ray diffraction analysis for the crystalline state of the polymorphic form A1 of the clopidogrel (−)-(1R)-camphor-10-sulfonate showed that it was a crystal having the characteristic diffraction pattern as shown in FIG. 1. The main diffraction peaks having I/I₀ value greater than 20%, are listed in Table 1.

TABLE 1

| 2θ (±0.2) | d (±0.2) | I/I₀ (%) | 2θ (±0.2) | d | I/I₀ (%) |
|---|---|---|---|---|---|
| 8.2 | 10.7 | 44.7 | 20.6 | 4.3 | 92.8 |
| 8.7 | 10.1 | 48.4 | 22.7 | 3.9 | 92.9 |
| 11.2 | 7.9 | 100.0 | 23.9 | 3.7 | 29.7 |
| 16.4 | 5.4 | 58.9 | 24.3 | 3.7 | 38.4 |
| 17.0 | 5.2 | 77.9 | 26.2 | 3.4 | 37.0 |
| 18.6 | 4.8 | 70.0 | 27.3 | 3.3 | 33.6 |
| 19.1 | 4.6 | 55.7 | 36.9 | 2.4 | 21.1 |

2θ: angle of diffraction, d: distance within each crystal face, I/I₀ (%): relative intensity of peak

### Example 2

### Preparation of clopidogrel (+)-(1S)-camphor-10-sulfonate (Polymorphic Form B1)

20 g of clopidogrel free base having an optical purity of 98.5% ee was dissolved in 200 ml of methyl ethyl ketone, and 14.5 g of (+)-(1S)-camphor-10-sulfonic acid was added thereto. The mixture was stirred at room temperature for 30 minutes, and then, at a temperature ranging from 0 to 5° C. for 4 hours. The precipitates formed were filtered, washed with cool methyl ethyl ketone and dried at 50° C., to obtain 29.6 g of the title compound (yield: 86%) as an white crystal.

m.p.: 149-150° C.

water (Karl-Fisher titrator): below 0.2%

specific rotatory power: [α]_{D}²⁰+60.2 (c=1, methanol)

optical purity (HPLC): above 99.6% ee

IR (KBr, cm⁻¹): 2959, 2936, 1752, 1739, 1452, 1437, 1301, 1241, 1147, 1028, 753, 723, 613 (FIG. 6)

DSC (10° C./min): starting point about 145° C., lowest point about 151° C. (heat absorption about 70 J/g, FIG. 5)

The result of powder X-ray diffraction analysis for the crystalline state of the polymorphic form B1 of the clopidogrel (+)-(1S)-camphor-10-sulfonate showed that it was a crystal having the characteristic diffraction pattern as shown in FIG. 4. The main diffraction peaks having I/I₀ value greater than 20%, are listed in Table 2.

TABLE 2

| 2θ (±0.2) | d (±0.2) | I/I₀ (%) | 2θ (±0.2) | d | I/I₀ (%) |
|---|---|---|---|---|---|
| 8.3 | 10.6 | 100.0 | 18.3 | 4.9 | 39.0 |
| 11.5 | 7.7 | 20.1 | 18.9 | 4.7 | 74.6 |
| 13.0 | 6.8 | 27.5 | 19.8 | 4.5 | 39.4 |
| 13.3 | 6.7 | 69.8 | 21.4 | 4.2 | 29.4 |
| 14.1 | 6.3 | 23.3 | 22.4 | 4.1 | 37.2 |
| 15.0 | 5.9 | 34.7 | 25.1 | 3.5 | 43.1 |
| 17.1 | 5.2 | 21.1 | | | |

2θ: angle of diffraction, d: distance within each crystal face, I/I₀ (%): relative intensity of peak

### Example 3

### Preparation of clopidogrel (+)-(1S)-camphor-10-sulfonate (Polymorphic Form B1)

1 g of the polymorphic form B1 of the clopidogrel (+)-(1S)-camphor-10-sulfonate (clopidogrel camsilate) obtained in Example 2 was dissolved in 12 ml of acetone under a reflux. The solution was cooled to room temperature and a small quantity of the polymorphic form B1 was added thereto. The mixture was stirred at room temperature for 2 hours, and then, at a temperature ranging from 0 to 5° C. for 12 hours. The precipitates formed were filtered, washed with cool acetone and dried at 50° C., to obtain 0.75 g of the title compound (yield: 75%) as an white crystal.

### Example 4

### Preparation of clopidogrel (+)-(1S)-camphor-10-sulfonate (Polymorphic Form B1)

1 g of clopidogrel free base was dissolved in 10 ml of ethyl acetate, and 0.72 g of (+)-(1S)-camphor-10-sulfonic acid was added thereto. The mixture was stirred at room temperature for 1 hour, and then, at a temperature ranging from 0 to 5° C. for 4 hours. The precipitates formed were filtered, washed with cool ethyl acetate and dried at 50° C., to obtain 1.55 g of the title compound (yield: 90%) as an white crystal.

### Example 5

### Preparation of clopidogrel (+)-(1S)-camphor-10-sulfonate (Polymorphic Form B1)

1 g of clopidogrel free base was dissolved in 10 ml of methyl isobutyl ketone, and 0.72 g of (+)-(1S)-camphor-10-sulfonic acid was added thereto. The mixture was stirred at room temperature for 1 hour, and then, at a temperature ranging from 0 to 5° C. for 4 hours. The precipitates formed were filtered, washed with cool methyl isobutyl ketone and dried at 50° C., to obtain 1.39 g of the title compound as an white crystal.

### Example 6

### Preparation of clopidogrel (+)-(1S)-camphor-10-sulfonate (Polymorphic Form B1)

1 g of clopidogrel free base was dissolved in 10 ml of acetone, and 0.72 g of (+)-(1S)-camphor-10-sulfonic acid and 10 ml of isopropyl ether were added thereto. The mixture was stirred at room temperature for 2 hours, and then, at a temperature ranging from 0 to 5° C. for 4 hours. The precipitates formed were filtered, washed with a mixture of acetone and isopropyl ether, and dried at 50° C., to obtain 1.52 g of the title compound (yield: 88%) as an white crystal.

### Example 7

### Preparation of clopidogrel (+)-(1S)-camphor-10-sulfonate (Polymorphic Form B1)

1 g of clopidogrel free base was dissolved in 10 ml of acetone, and 0.72 g of (+)-(1S)-camphor-10-sulfonic acid and 10 ml of methyl t-butyl ketone were added thereto. The mixture was stirred at room temperature for 2 hours, and then, at a temperature ranging from 0 to 5° C. for 4 hours. The precipitates formed were filtered, washed with a mixture of acetone and methyl t-butyl ketone, and dried at 50° C., to obtain 1.45 g of the title compound (yield: 84%) as an white crystal.

### Example 8

### Preparation of clopidogrel (+)-(1S)-camphor-10-sulfonate (Polymorphic Form B2)

0.5 g of clopidogrel free base was dissolved in 2 ml of methyl ethyl ketone, and 15 [symbol] of methyl t-butyl ether were immediately added thereto. The resulting gel mixture was allowed to be solidified while scraping with a spatula. The solidified crystals were filtered, washed with methyl t-butyl ether and dried with a nitrogen flow, to obtain 0.6 g of the title compound (yield: 70%) as an white powder.

m.p.: 135-136° C.

water (Karl-Fisher titrator): below 0.2%

specific rotatory power: [α]_{D}²⁰+60.2 (c=1, methanol)

optical purity (HPLC): above 99.5% ee

IR (KBr, cm⁻¹): 2959, 1748, 1479, 1446, 1322, 1301, 1233, 1201, 1145, 1024, 754, 725, 614 (FIG. 9)

DSC (10° C./min): starting point about 132° C., lowest point about 138° C. (heat absorption about 54 J/g, FIG. 8)

The result of powder X-ray diffraction analysis for the crystalline state of the polymorphic form B2 of the clopidogrel (+)-(1S)-camphor-10-sulfonate showed that it was a crystal having the characteristic diffraction pattern as shown in FIG. 7. The main diffraction peaks having I/I₀ value greater than 10%, are listed in Table 3.

TABLE 3

| 2θ (±0.2) | d (±0.2) | I/I₀ (%) | 2θ (±0.2) | d | I/I₀ (%) |
|---|---|---|---|---|---|
| 7.9 | 9.9 | 100.0 | 17.8 | 5.0 | 37.6 |
| 11.9 | 7.5 | 12.5 | 19.2 | 4.6 | 13.7 |
| 14.1 | 6.3 | 10.5 | 19.5 | 4.5 | 45.5 |
| 15.1 | 5.9 | 13.9 | 20.9 | 4.3 | 12.1 |
| 15.7 | 5.6 | 13.9 | 24.9 | 3.6 | 19.5 |
| 17.5 | 5.1 | 23.6 | | | |

2θ: angle of diffraction, d: distance within each crystal face, I/I₀ (%): relative intensity of peak

### Example 9

### Preparation of clopidogrel (+)-(1S)-camphor-10-sulfonate (Polymorphic Form B2)

1 g of clopidogrel free base was dissolved in 10 ml of ethyl acetate, and 0.72 g of (+)-(1S)-camphor-10-sulfonic acid and 10 mg of the polymorphic form B2 obtained in Example 8 were added thereto. Then, the mixture was quickly cooled to a temperature ranging from 0 to 5° C., and then, it was kept at that temperature for one night while intermittently stirring. The precipitates formed were filtered, washed with cool ethyl acetate and dried at 50° C., to obtain 1.43 g of the title compound (yield: 83%) as an white crystal.

### Example 10

### Preparation of clopidogrel (+)-(1S)-camphor-10-sulfonate (Polymorphic Form B3)

1 g of clopidogrel (+)-(1S)-camphor-10-sulfonate was dissolved in 5 ml of methanol, and the solution was evaporated under a reduced pressure to remove the solvent therefrom. 15 ml of n-pentane was added to the foam residue and stirred for homogenization. The resulting solid was filtered, washed with n-pentane and dried with a nitrogen flow, to obtain 1.3 g of the title compound (yield: 76%) as an white powder.

m.p.: starting at 65° C. and completed at 110° C.

water (Karl-Fisher titrator): below 0.5%

optical purity (HPLC): 99.0% ee

IR (KBr, cm⁻¹): 2956, 1745, 1479, 1438, 1229, 1147, 1035, 761, 615

DSC (10° C./min): no prominent heat absorption and discharge peaks (FIG. 11)

The result of powder X-ray diffraction analysis for the crystalline state of the polymorphic form B3 of the clopidogrel (+)-(1S)-camphor-10-sulfonate showed that it was amorphous having no characteristic diffraction pattern as shown in FIG. 10.

### Test Example 1

### Optical Purity Measurement of Acid Addition Salt of Clopidogrel

Clopidogrel free bases having optical purities of about 90% ee, 95% ee and 98.5% ee, respectively, were prepared, and, clopidogrel hydrogen sulfates and clopidogrel (+)-(1S)-camphor-10-sulfonates were prepared using the clopidogrel free bases according to the procedures described in Comparative Example 1 and Example 2, respectively. The optical purities of the acid addition salts obtained were measured under HPLC condition C, and the extents of optical purity improvement are shown in Table 4.

TABLE 4

| Starting material | | |
|---|---|---|
| Clopidogrel free | Obtained acid addition salt of clopidogrel | |
| base | Hydrogen sulfate | (+)-(1S)-camphor-10-sulfonate |
| 90.0 | 89.7 | 98.5 |
| 95.0 | 95.1 | 99.2 |
| 98.5 | 98.3 | 99.6 |

Unit: % ee (the excess amount of enantiomers)

As shown in Table 4, the optical purity of the clopidogrel camphorsulfonate according to the present invention was markedly enhanced during the process of preparing same, while that of clopidogrel hydrogen sulfate was not improved.

Clopidogrel is liable to be partially racemized to its levorotatory isomer, and thus, a plurality of purification steps is required to achieve a pharmaceutically acceptable optical purity. However, the inventive clopidogrel camphorsulfonate meets the pharmaceutical optical purity requirements without separate purification steps.

### Test Example 2

### Water-Solubility Measurement of Acid Addition Salt of Clopidogrel

The camphorsulfonate polymorphic form A1 obtained in Example 1, the camphorsulfonate polymorphic form B1 obtained in Example 2, the hydrogen sulfate obtained in Comparative Example 1 and the benzenesulfonate obtained in Comparative Example 2 were each dissolved in unionized water until saturated. The amounts of clopidogrel in the resulting solutions were measured under HPLC condition A, and the results are shown in Table 5.

TABLE 5

| | Water-solubility |
|---|---|
| Acid addition salt of clopidogrel | (mg/ml, as clopidogrel) |
| Camphorsulfonate (polymorphic form B1) | 3.98 |
| Camphorsulfonate (polymorphic form A1) | 4.04 |
| Hydrogen sulfate | 7.42 |
| Benzenesulfonate | 4.74 |

As shown in Table 5, the water-solubility of the clopidogrel camphorsulfonate according to the present invention was lower than that of hydrogen sulfate or benzenesulfonate, but such water-solubility level is more than sufficient for use in a pharmaceutical composition.

### Test Example 3

### Stability Test of Acid Addition Salt of Clopidogrel Under Moist and Heated Condition

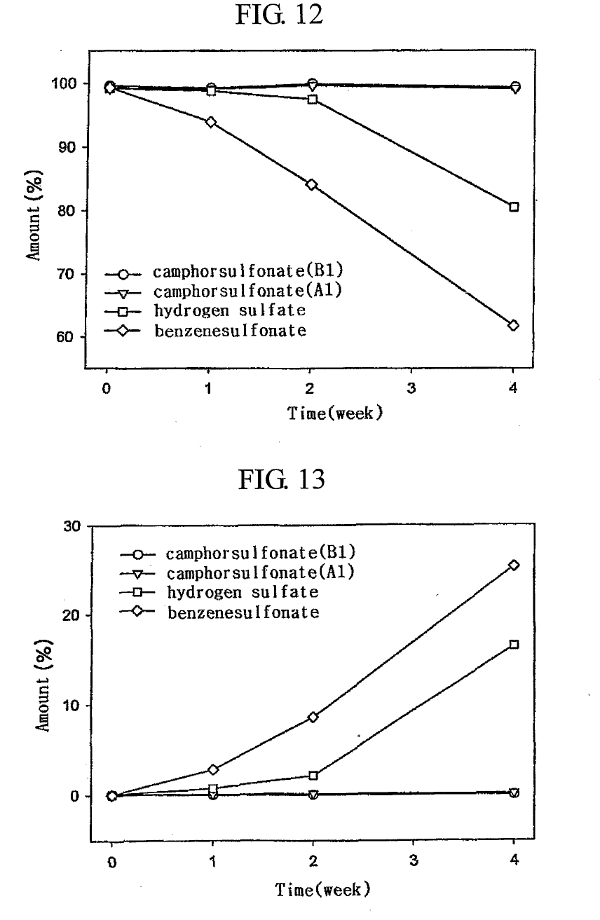

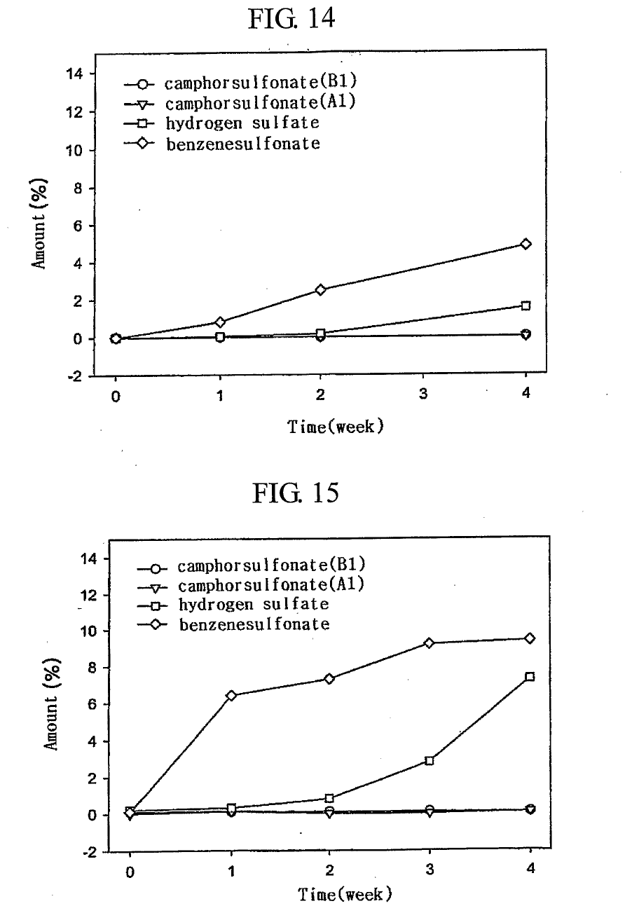

The camphorsulfonate polymorphic form A1 obtained in Example 1, the camphorsulfonate polymorphic form B1 obtained in Example 2, the hydrogen sulfate obtained in Comparative Example 1 and the benzenesulfonate obtained in Comparative Example 2 were each subjected to a condition of 60±2° C. and 75±5% relative humidity for a period of 28 days to test their stabilities. The changes of the assay of the acid addition salts of clopidogrel, and the amounts of hydrolyzed impurities and levorotatory isomers thereof and the water content thereof were measured at 7, 14, 21 and 28 days relative to that of the initial day (0) using HPLC. The results are shown in Tables 6 to 9 and FIGS. 12 to 15, respectively.

TABLE 6

| | Amount of acid addition | | | |
|---|---|---|---|---|
| | salts of clopidogrel (%) | | | |
| | 0 day | 7 days | 14 days | 28 days |
| Camphorsulfonate | 99.6 | 99.2 | 99.9 | 99.3 |
| (polymorphic form B1) | | | | |
| Camphorsulfonate | 99.2 | 99.1 | 99.6 | 99.1 |
| (polymorphic form A1) | | | | |
| Hydrogen sulfate | 99.2 | 98.8 | 97.5 | 80.5 |
| Benzenesulfonate | 99.3 | 93.9 | 80.5 | 61.7 |

TABLE 7

| | Amount of hydrolyzed impurities (%) | | | |
|---|---|---|---|---|
| | 0 day | 7 days | 14 days | 28 days |
| Camphorsulfonate | 0.01 | 0.01 | 0.02 | 0.03 |
| (polymorphic form B1) | | | | |
| Camphorsulfonate | 0.01 | 0.08 | 0.15 | 0.17 |
| (polymorphic form A1) | | | | |
| Hydrogen sulfate | 0.03 | 0.72 | 2.15 | 16.49 |
| Benzenesulfonate | 0.01 | 2.79 | 8.56 | 25.33 |

TABLE 8

| | Amount of levorotatory isomers (%) | | | |
|---|---|---|---|---|
| | 0 day | 7 days | 14 days | 28 days |
| Camphorsulfonate | 0.01 | 0.01 | 0.02 | 0.03 |
| (polymorphic form B1) | | | | |
| Camphorsulfonate | 0.02 | 0.02 | 0.02 | 0.02 |
| (polymorphic form A1) | | | | |
| Hydrogen sulfate | 0.01 | 0.05 | 0.19 | 1.53 |
| Benzenesulfonate | 0.01 | 0.81 | 2.49 | 4.79 |

TABLE 9

| | Water content (%) | | | | |
|---|---|---|---|---|---|
| | 0 day | 7 days | 14 days | 21 days | 28 days |
| Camphorsulfonate | 0.1 | 0.1 | 0.1 | 0.1 | 0.1 |
| (polymorphic form B1) | | | | | |
| Camphorsulfonate | 0.0 | 0.1 | 0.0 | 0.0 | 0.1 |
| (polymorphic form A1) | | | | | |
| Hydrogen sulfate | 0.2 | 0.3 | 0.8 | 2.8 | 7.3 |
| Benzenesulfonate | 0.1 | 6.4 | 7.3 | 9.2 | 9.4 |

As shown in Tables 6 to 9 and FIGS. 12 to 15, the inventive clopidogrel camphorsulfonate and its polymorphic forms were less hygroscopic, and more stable against moisture or heat, as compared to hydrogen sulfate and benzenesulfonate, which suggests that there is no significant decline in the amount of optically pure clopidogrel even after storage under a severe condition for a long period. These results confirm that a pharmaceutical composition comprising the inventive clopidogrel camphorsulfonate is more stable than conventional acid addition salts.

The inventive clopidogrel camphorsulfonate and its polymorphic forms may be formulated alone or in a combination with pharmaceutically acceptable additives, according to any of the conventional methods used to prepare soft or hard capsules and tablets.

The following Preparation Examples are intended to further illustrate the present invention without limiting its scope.

### Preparation Example 1

### Soft or Hard Capsule

A gelatin capsule was prepared using the following ingredients:

| | Quantity(mg/capsule) |
|---|---|
| Clopidogrel camphorsulfonate | 129 |
| (polymorphic form B1) | |
| Lactose | 90 |
| Corn starch | 25 |
| Silicon dioxide colloid | 4 |
| Magnesium stearate | 2 |
| Total | 250 |

### Preparation Example 2

### Tablet

A tablet was prepared using the following ingredients:

| | Quantity(mg/tablet) |
|---|---|
| Clopidogrel camphorsulfonate | 129 |
| (polymorphic form B1) | |
| Anhydrous lactose | 90 |
| Microcrystalline cellulose | 20 |
| Hydroxypropylcellulose | 6 |
| Polysorbate | 2 |
| Hydrogenated castor oil | 1 |
| Magnesium stearate | 1 |
| Solid polyethylene glycol | 1 |
| Total | 250 |

As discussed above, the inventive clopidogrel camphorsulfonate and its polymorphic forms are very stable against moisture and heat. Accordingly, the inventive pharmaceutical composition comprising same can maintain a high purity of an active ingredient for a prolonged time, and it can be very useful in the prevention and treatment for platelet aggregation-associated diseases.

While the invention has been described with respect to the above specific embodiments, it should be recognized that various modifications and changes may be made to the invention by those skilled in the art which also fall within the scope of the invention as defined by the appended claims.

1-9. (canceled)

10. A polymorphic form of clopidogrel (+)-(1S)-camphor-10-sulfonate of formula (Ib):

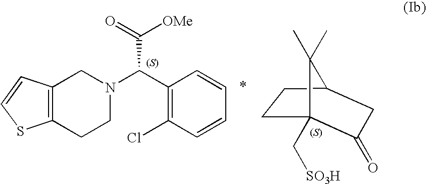

11. The polymorphic form of clopidogrel (+)-(1S)-camphor-10-sulfonate of claim 10, which is the polymorphic form B1 whose powder X-ray diffraction scan shows major peaks having I/I₀ values greater than 20% at 2θ of 8.3, 11.5, 13.0, 13.3, 14.1, 15.0, 17.1, 18.3, 18.9, 19.8, 21.4, 22.4, 25.1 and 26.0.

12. The polymorphic form of clopidogrel (+)-(1S)-camphor-10-sulfonate of claim 10, which is the polymorphic form B2 whose powder X-ray diffraction scan shows major peaks having I/I₀ values greater than 10% at 2θ of 7.9, 11.9, 14.1, 15.1, 15.7, 17.5, 17.8, 19.2, 19.5, 20.9 and 24.9.

13. The polymorphic form of clopidogrel (+)-(1S)-camphor-10-sulfonate of claim 10, which is the polymorphic form B3 which is amorphous.

14. A pharmaceutical composition for treating or preventing a platelet aggregation-associated disease, which comprises the polymorphic form of clopidogrel (+)-(1S)-camphor-10-sulfonate according to claim 10 as an active ingredient.

15. The pharmaceutical composition of claim 14, wherein the platelet aggregation-associated disease is stroke, cerebral arteriosclerosis, myocardial infarction, angina pectoris, arrhythmia, peripheral arteries disease or Burger's disease.

16. The pharmaceutical composition of claim 14, which is orally administered.

17. The pharmaceutical composition of claim 14, wherein the polymorphic form of clopidogrel (+)-(1S)-camphor-10-sulfonate is present in an amount ranging from 0.1 to 95% by weight based on the total weight of the composition.

18. The pharmaceutical composition of claim 17, wherein the polymorphic form of clopidogrel (+)-(1S)-camphor-10-sulfonate is present in an amount ranging from 1 to 70% by weight based on the total weight of the composition.

19. A pharmaceutical composition for treating or preventing a platelet aggregation-associated disease, which comprises the polymorphic form of clopidogrel (+)-(1S)-camphor-10-sulfonate according to claim 11 as an active ingredient.

20. A pharmaceutical composition for treating or preventing a platelet aggregation-associated disease, which comprises the polymorphic form of clopidogrel (+)-(1S)-camphor-10-sulfonate according to claim 12 as an active ingredient.

21. A pharmaceutical composition for treating or preventing a platelet aggregation-associated disease, which comprises the polymorphic form of clopidogrel (+)-(1S)-camphor-10-sulfonate according to claim 13 as an active ingredient.
